(19) United States
(12) Patent Application Publication
(10) Pub. No.: US 2017/0060298 A1
(43) Pub. Date: Mar. 2, 2017

(54) Smart Interaction Device

(71) Applicant: Futureplay, Inc., Seoul (KR)

(72) Inventors: Sungjae Hwang, Seoul (KR); Jongho Kim, Seoul (KR)

(21) Appl. No.: 15/010,100

(22) Filed: Jan. 29, 2016

(30) Foreign Application Priority Data
Aug. 26, 2015 (KR) 10-2015-0120231
Aug. 26, 2015 (KR) PCT/KR2015/013337

(51) Int. Cl. G06F 3/044; A61B 5/00; A41D 1/00

(52) U.S. Cl. CPC G06F 3/044; A41D 1/005; A61B 5/6804

(57) ABSTRACT

Accordingly the embodiments herein provides a smart interaction system including a fabric device comprising a conductive thread, a sensing area including a partial area of the conductive thread, and a sensor. Further, smart interaction system includes a control module of a mobile device configured to be detachably connected to the fabric device and to communicate with the fabric device, wherein the mobile device including a touch interface configured to receive a first touch input provided to the sensing area. The sensing area of the fabric device communicates with the mobile device by using the conductive thread. The mobile device is driven by using a second touch input provided to the touch interface based on the first touch input.

### CROSS-REFERENCE TO RELATED APPLICATIONS

This application claims priority to International Application No. PCT/KR2015/013337, filed on Aug. 26, 2015; and Korea Application No. 10-2015-0120231, filed on Aug. 26, 2015, from which the International Application claims priority. The aforementioned applications are incorporated herein by reference.

### TECHNICAL FIELD

The present invention relates to a portable electronic device for data transfer. More particularly, related to a mechanism for exchanging sensor data between a garment provided with a conductive thread and one or more external devices electromagnetically connected to the garment.

### BACKGROUND

Research on a method for data transmission utilizing a smart garment or wearable devices has been actively conducted. The smart garment is a garment manufactured by using a conductive thread which serves as a means for transmitting information of various sensors provided in the garment to a controller.

In addition to the conductive thread, the smart garment is provided with a battery, a controller, a wired/wireless communication means. When the smart garment is washed, the battery, the controller, the wired/wireless communication means or the like has to be removed from the smart garment. Thus, the conventional smart garment are non-washable and cannot replace the non-smart garments in terms of usual use. Accordingly, a washable smart garment without removing the battery, the controller, the wired/wireless communication means or the like have been acutely demanded.

Further, as a user wears a garment throughout a day, the garment can be used to recognize various context information such as a user location, a user posture, an emotional state of the user, or the like. Thus, there remains a need of mechanism for recognizing such context information of the user through a smart garment, and utilizing the recognized context information through smart devices to perform various actions.

### SUMMARY OF INVENTION

A technical problem of the present invention is to provide a simple and robust mechanism for recognizing context information of a user through a smart garment (also referred as a fabric device), and utilizing the recognized context information through external devices to perform various actions. Various challenges are raised to configure the fabric device and an external device into one platform through an electromagnetical connection between the fabric device and the external device. The external device is either in contact or in close proximate to the fabric device. The fabric device comprises a conductive thread to exchange sensor information of various sensors provided in the fabric device with the external device.

In order to achieve the technical problem, an embodiment of the present invention discloses a smart interaction system including a fabric device. The fabric device including a conductive thread, a non-conductive thread, and a sensing area including at least a partial area of the conductive thread and at least a partial area of the non-conductive thread. Further, the fabric device includes a first control module configured to sense one of a resistance and capacitive change of the partial area of the conductive thread, generate at least one of a first signal and a second signal based on one of the resistance change and the capacitive change, wherein the first signal is different from the second signal; and send at least one of the first signal and the second signal to a mobile device.

In an embodiment, the fabric device further includes a second control module configured to control the mobile device to be operated in a first mode when receiving the first signal, and to be operated in a second mode different from the first mode when receiving the second signal.

In an embodiment, the first control module further configured to determine whether the user takes a first action based on the resistance change and generate the first signal; and determine whether the user takes a second action based on one of the resistance and capacitance change and generate the second signal.

In an embodiment, the first action includes sleeping, moving, standing, exercising and eating, wherein the second action includes waking, stopping a movement, breaking an exercise, and stopping to eat.

In an embodiment, the sensing area includes a touch sensing area including a stitched pattern of the conductive thread.

In an embodiment, the fabric device further includes an interface which includes a case which accommodates a main body of a watch and a first connector, wherein the fabric device includes a second connector and the first connector, wherein the second connector is detachably connected.

In an embodiment, the interface further includes the first and second control modules.

Accordingly the embodiments herein provides a smart interaction system including a fabric device comprising a conductive thread, a sensing area including a partial area of the conductive thread, and a sensor. Further, smart interaction system includes a control module of a mobile device configured to be detachably connected to the fabric device and to communicate with the fabric device, wherein the mobile device including a touch interface configured to receive a first touch input provided to the sensing area. The sensing area of the fabric device communicates with the mobile device by using the conductive thread. The mobile device is driven by using a second touch input provided to the touch interface based on the first touch input.

In an embodiment, the touch interface includes a first area and a second area, and the sensing area includes a third area physically corresponding to the first area and a fourth area physically corresponding to the second area.

In an embodiment, the touch input sensed in the third area is recognized as an input to the first area of the touch interface, and the touch input sensed in the fourth area is recognized as an input to the second area of the touch interface.

In an embodiment, the touch interface includes a first area coupled to a function in a first mode of the mobile device and a second area which is different from the first area and coupled to the function in a second mode;

In an embodiment, the sensing area includes a third area physically corresponding to the second area;

In an embodiment, when the mobile device connected to the fabric device, the mobile device is operated in the first mode and when the mobile device disconnected to the fabric device, the mobile device is operated in the second mode; and

In an embodiment, the touch input sensed in the third area is recognized as an input to the second area of the touch interface in the second mode.

In an embodiment, the fabric device is further configured to determine whether the user takes a first action based on the resistance change which is sensed by the conductive thread; and generate a first signal which represents a first state of the fabric device.

In an embodiment, the mobile device is further configured to operate the mobile device in a first mode when the first state is determined.

In an embodiment, the fabric device is further configured to determine whether the user takes a second action based on the resistance change; and generate a second signal which represents a second state of the fabric device.

In an embodiment, the mobile device is further configured to operate the mobile device in a second mode when the second state is determined.

Accordingly the embodiments herein provides a mobile device configured to communicate with a fabric device comprising a touch interface configured to receive a touch input provided to a sensing area of the fabric device. Further, the mobile device includes a controller unit configured to receive at least one of a first signal and a second signal from the fabric device, wherein the first signal is different from the second signal; and operate in one of a first mode when the first signal is received, and operate in a second mode when the second signal is received, wherein the first mode is different from the second mode.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE FIGURES

This invention is illustrated in the accompanying drawings, throughout which like reference letters indicate corresponding parts in the various figures. The embodiments herein will be better understood from the following description with reference to the drawings, in which:

FIG. 1 is a block diagram of a smart interaction device, according to the embodiments as disclosed herein;

FIG. 2 is another block diagram of a smart interaction device, according to the embodiments as disclosed herein;

FIGS. 3A to 3C are diagrams illustrating characteristics of a touch input according to a conductive thread pattern and a touch reaction to a display of an external device electromagnetically connected with a conductive thread, according to the embodiments as disclosed herein;

FIGS. 4A and 4B are diagrams illustrating a characteristic of sensing information received by a sensor electrode, which is in contact with or in proximate to skin, according to the embodiment as disclosed herein;

FIGS. 5A and 5B are diagrams illustrating a form of an interface through which a mobile device and a fabric device may transmit and receive a signal, according to the embodiments as disclosed herein;

FIG. 6 is a diagram illustrating contents of sensing a pattern change in movements of a wearer by the garment to switch an operation mode of a smart watch, according to the embodiments as disclosed herein;

FIG. 7 is a diagram illustrating contents of a conductive thread at a position, at which a joint region is bonded, in order to sense a movement of a wearer of the garment, according to the embodiments as disclosed herein;

FIG. 8 is a diagram illustrating a conductive thread having elasticity, which is provided in a predetermined region of a garment in order to sense a movement of the wearer, according to the embodiments as disclosed herein;

FIG. 9 is a diagram illustrating an example scenario in which a touch input to an external device which is in contact with or in proximity to a garment, according to the embodiments as disclosed herein;

FIG. 10 is a diagram illustrating a virtual touch area of a sleeve of a garment and a touch input to a display of a smart watch, in a state where a connection terminal of the sleeve of the garment is physically or electromagnetically connected with a connection terminal of the smart watch, according to the embodiments as disclosed herein;

FIG. 11 is a diagram illustrating characteristics of various body movements and biometric information detected by an underwear, according to the embodiments disclosed herein;

FIG. 12 illustrates a characteristic of transmitting voice information reproduced by an external device to a wearer through a bone conduction speaker provided at a collar of the garment, according to the embodiments as disclosed here;

FIG. 13 is a diagram illustrating a characteristic of transceiving sensing information with a shoe which is in contact with or in proximity to a garment, according to the embodiments as disclosed herein;

FIG. 14 is a diagram illustrating a characteristic of transceiving sensing information with a glove which is in contact with or in proximity to a garment, according to the embodiments as disclosed herein;

FIG. 15 is a diagram illustrating a characteristic of transceiving information between a garment and a wearable device which is in contact with or in proximity to the garment, according to the embodiments as disclosed herein;

FIG. 16 is a diagram illustrating an example scenario in which contents of a contact is exchanged between a garment and an external object, according to the embodiments as disclosed herein;

FIG. 17 is a diagram illustrating a characteristic of a contact and a control characteristic between a garment and an accessory, according to the embodiments as disclosed herein;

FIG. 18 is a diagram illustrating an interaction characteristic by various bonding methods between a specific region of a garment and an external device, according to the embodiments disclosed herein;

FIG. 19 is a diagram illustrating contents of the different configuration between a touch pattern input into an external device and a touch pattern input into a conductive thread with respect to the same event, according to the embodiments as disclosed herein;

FIG. 20 is a diagram illustrating a characteristic of switching an operation mode of an external device in a state where a conductive thread of a garment is in contact with and electromagnetically connected with the worn external device, according to the embodiments as disclosed herein;

FIGS. 21A and 21B are diagrams illustrating a connection of a connection terminal positioned inside a sleeve of a fabric device with a connection terminal of a mobile device, according to the embodiments as disclosed herein;

FIGS. 22A and 22B are diagrams illustrating a configuration, in which the mobile device 200, which is configured to be detachable in a clip form, is coupled with the fabric device 100, according to the embodiments as disclosed herein;

FIG. 23 is a diagram illustrating a connection terminal of the fabric device and a connection terminal of a strap of the mobile device which are implemented in forms of a protrusion and a fitting hole respectively, according to the embodiments as disclosed herein;

FIG. 24 is a diagram illustrating connectors provided with connection terminals, which are in a form of a snap button, and have improved coupling force by an elastic member, according to the embodiments as disclosed herein; and

FIG. 25 is a diagram illustrating a connection of the connector of the FIG. 24 to a fabric device and a conductive thread, according to the embodiments as disclosed herein.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

Accordingly the embodiments herein disclose a fabric device including a conductive thread, a non-conductive thread, a sensing area including a partial area of the conductive thread and a partial area of the non-conductive thread. Further, the fabric device includes a controller unit is configured to sense a resistance change of the partial area of the conductive thread. Further, the controller unit is configured to generate at least one of a first signal and a second signal based on the resistance change, where the first signal is different from the second signal. Further, the controller unit is configured to send at least one of the first signal and the second signal to a mobile device.

Accordingly the embodiments herein disclose a smart interaction device. The smart interaction device includes a fabric device including a conductive thread, a sensing area including a partial area of the conductive thread, and a sensor. Further, smart interaction device includes a mobile device configured to communicate with the fabric device, wherein the mobile device including a touch interface configured to receive a touch input provided to the sensing area. The sensing area of the fabric device communicates with the mobile device by using the conductive thread and the mobile device is driven by using at least one of a resistance change of the partial conductive thread and a first touch input provided to the touch interface.

Accordingly the embodiments herein disclose a mobile device configured to communicate with a fabric device, including a touch interface configured to receive a touch input provided to a sensing area of the fabric device and a controller unit. The controller unit is configured to receive at least one of a first signal and a second signal from the fabric device, wherein the first signal is different from the second signal. Further, the controller unit is configured to operate in one of a first mode when the first signal is received, and operate in a second mode when the second signal is received, wherein the first mode is different from the second mode.

Unlike the conventional devices and methods, a simple and robust mechanism for recognizing context information of a user through the fabric device (garment) and utilizing the recognized context information through external devices to perform various actions is disclosed. Various challenges are raised to configure the fabric device and an external device into one platform through an electromagnetical connection between the fabric device and the external device. According to the present invention, even though hardware, such as a separate processor (CPU), memory, and battery, essential to the computing is not directly mounted or provided in the garment, the garment may become a device using an electromagnetic connection with an external device, which is in contact with or in proximate to the garment. The external device is either in contact or in close proximate to the fabric device. The fabric device comprises a conductive thread to exchange sensor information of various sensors provided in the fabric device with the external device. In this respect, when the garment is mass-produced, it is possible to reduce manufacturing cost, decrease weight of the garment to provide a user with a comfortable garment.

Further, it is possible to easily achieve an original object, such as washing, of a garment, and provide one platform, in which the garment is capable of interacting with the external device, thereby providing a user with new user experiences.

Further, it is possible to provide a user with new experiences by sensing various context information through a smart garment.

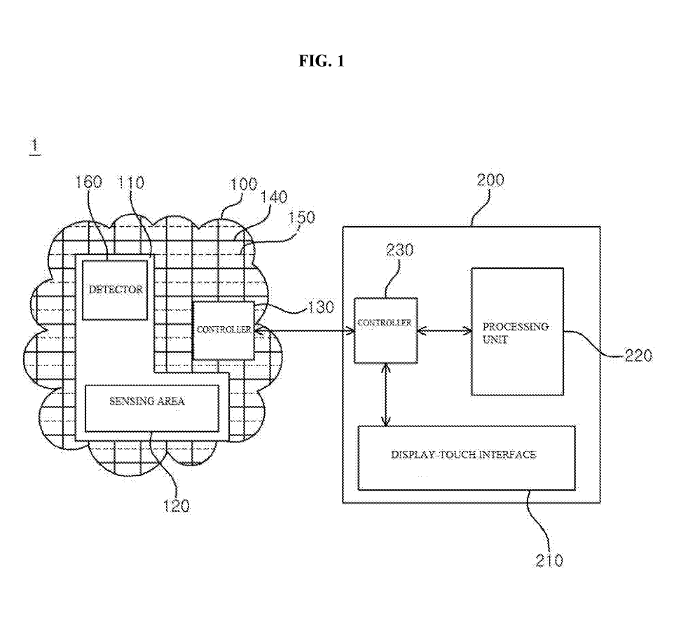

Referring now to the drawings and more particularly to FIGS. 1 to 25 where similar reference characters denote corresponding features consistently throughout the figures, there are shown preferred embodiments.

FIG. 1 is a block diagram of a smart interaction device 1, according to the embodiments as disclosed herein. In an embodiment, the smart interaction device 1 may include a fabric device 100 and a mobile device 200.

The fabric device 100 may include a device formed of a fabric material made of an electronic thread or a conductive thread, which is capable of transporting an electronic material or being charged.

Otherwise, according to another embodiment of the present invention, the fabric device 100 may be configured to include a pattern or a network formed of an electronic thread or a conductive thread and can be operated by utilizing a controller of a mobile device. For example, a touch pattern form of an electronic thread or a conductive thread may be included in the fabric device 100. The fabric device 100 may be configured to be connected with the mobile device 200 through a connector.

In several embodiments of the present invention, the fabric device 100 may include a garment device, which is detachable by a user. The garment device may be configured to surround at least a part of a body of the user with a plurality of surfaces thereof. In an embodiment, the garment device can be in close contact with at least a part of a body of the user. For example, the garment device may be manufactured in various forms, such as a garment including a shirt, trousers, a skirt, a hat, a bag, or arm warmers. Otherwise, the garment device may be manufactured in the form of a bag and can be in close contact with a part of a body of the user.

The fabric device 100 may include a conductive thread 140 and a non-conductive thread 150 together. Particularly, the fabric device 100 may be formed by entangling the conductive thread 140 and the 150 with each other. In this case, the non-conductive thread 150 may serve as an insulator between the adjacent conductive threads 140.

Although the sewing of the conductive thread 140 and the non-conductive thread 150 in a vertical direction is conceptually illustrated in the drawing, the technical spirit of the present invention is not limited thereto. The conductive thread 140 and the non-conductive thread 150 may be implemented in various stitch patterns according to an entangled form thereof.

In several exemplary embodiments, the fabric device 100 may include a sensor 110. Although only one sensor 110 is illustrated in the drawing, the fabric device 100 may include a plurality of sensors 110.

When the fabric device 100 includes the plurality of sensors 110, the conductive thread 140 may serve to electrically connect the plurality of sensors 110.

The sensor 110 may serve to sense information associated with a user (or a wearer) of the fabric device 100. In several embodiments, the sensor 110 may serve to physically sense the user of the fabric device 100. For example, the sensor 100 may sense whether the user of the fabric device 100 is in a bent state or a stretched state. In another example, the sensor 110 may sense whether the user is in a running state or a walking state. More particular examples thereof will be described below.

In an embodiment, the sensor 110 may serve to biologically sense the user of the fabric device 100. For example, the sensor 100 may sense whether the user of the fabric device 100 has a high temperature or a low temperature.

In an embodiment, the sensor 110 may be coupled with an inner/outer side of the fabric device 100 to sense physical information, chemical information, biological information, or the like sensing information related to the wearer of the garment. Some non-limiting examples of the sensor 110 may include a touch sensor, a motion sensor, a bending sensor, a pressure sensor, a temperature sensor, a proximity sensor, a humidity sensor, a gas sensor, an electrocardiogram (ECG) sensor, a photoplethysmogram (PPG) sensor, an electroencephalogram (EEG) sensor, a pulse sensor, a breathing sensor, and an SPO2 sensor.

In an embodiment, a conductive surface of the conductive thread 140 may also serve as a virtual touch sensor for sensing touch input information. The conductive surface may also be defined as a sensing area 120, which will be described below.

In an embodiment, the sensor 110 may include the sensing area 120 and a detector 160. The sensing area 120 may include at least a partial area of the conductive thread 140 and at least a partial area of the non-conductive thread 150.

The sensing area 120 may sense a resistance change of the conductive thread 140 included in the sensing area 120 to perform the sensing. For example, when heat is generated at a part of the body of the user of the fabric device 100, which is in contact with the sensing area 120, a resistance change may be generated in the conductive thread 140 included in the sensing area 120, and the sensing area 120 may sense the resistance change.

Further, when sweat comes out from the part of the body of the user of the fabric device 100, which is in contact with the sensing area 120, a resistance change may be generated in the conductive thread 140 included in the sensing area 120, and the sensing area 120 may sense the resistance change.

Further, for example, when a finger of the user of the fabric device 100 is in contact with the sensing area 120, a resistance change may be generated in the conductive thread 140 included in the sensing area 120 or a capacitance change may be generated between the conductive threads 140. In this case, the sensing area 120, a controller 130 electrically connected with the sensing area 120, or a controller 230 of the mobile device 200 may sense the contact of the finger of the user. That is, the sensing area 120 may include a touch sensor sensing a touch input by using a pattern stitched by the conductive thread 140 or the non-conductive thread 150. The pattern may be stitched in the fabric device directly. Alternatively the pattern may be stitched in other clothes and patched to the fabric device. More particular examples thereof will be described below.

Although one sensing area is illustrated in the drawing, the sensing area 120 may be further added as many as possible.

When the fabric device 100 includes the plurality of sensing areas 120, the conductive thread 140 may serve to electrically connect the plurality of sensing areas 120.

Further, in several exemplary embodiments, when the fabric device 100 includes the plurality of sensing areas 120, the plurality of sensing areas 120 may have different stitched patterns by using the conductive thread 140 and the non-conductive thread 150. When the plurality of sensing areas 120 has the different stitched patterns, sensing efficiency may be further improved.

For example, the sensing area 120, which is in contact with an elbow of the user of the fabric device 100, may have a relatively loose stitch pattern, compared to the sensing area 120, which is in contact with a sole of the user of the fabric device 100, thereby further improving the sensing efficiency.

The detector 160 may detect an electrical change generated in the sensing area under the control of the controller 130. For example, when the sensor 110 is a touch sensor, the sensor 110 may sense a resistance change or a capacitance change in the sensing area 120. The sensor 110 may detect whether a change in a voltage value or a current value generated by the resistance change or the capacitance change is equal to or larger than a reference value. Further, the detector 160 transmit a result of the detection to the controller 130.

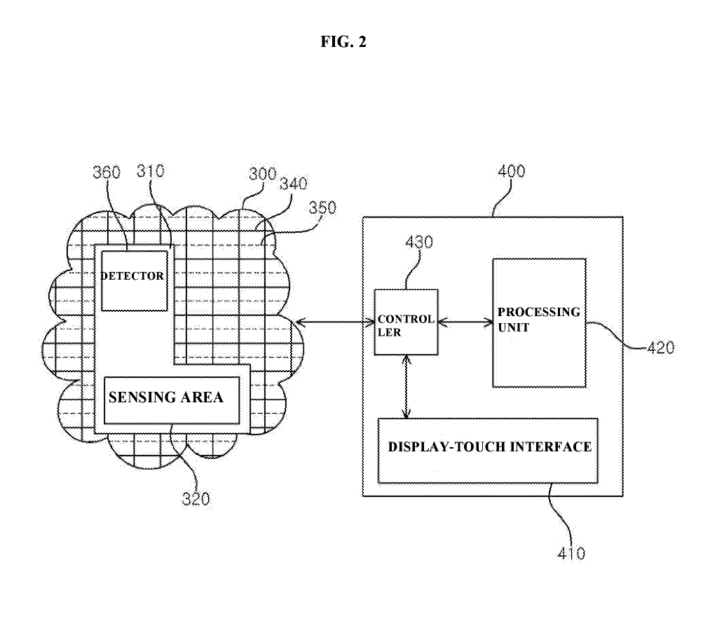

The detector 160 may also be implemented to be separate from the controller 130 as illustrated in FIGS. 1 and 2, and may also be included in the controller 130.

As illustrated in the FIG. 1, the sensing area 120 is provided in at least a partial region of the fabric device 100. For example, an area detecting a capacitance change may be implemented by implementing the conductive threads 140 to cross in a partial region to become an area sensing a touch of the user. In an embodiment, the sensing area 120 may sense a posture of the user or the like by sensing an electrical characteristic, such as resistance changed according to an increase or shrinkage of the conductive thread 140 when an elbow or a knee of a person is bent. In an embodiment, the sensing area 120 may sense an electrocardiogram change of the user by disposing the sensing area 120 at a chest portion of the user.

For example, the sensing area 120 may sense whether the mobile device 200 is accommodated in a specific pocket by providing the sensing area 120, which is capable of sensing a contact/proximity, within the pocket of the fabric device 100. In addition, it is possible to sense various context information of the user by sensing a posture of the user or a contact with the mobile device 200 by using various methods through various sensors.

The conductive thread 140 may be used for transmitting the sensing information sensed by the sensor 110 to the mobile device 200, which is in contact with or in proximity to the fabric device 100. Further, the conductive thread 140 itself may also be used as one of the sensors 110. The conductive thread 140 may replace a natural fiber forming a garment, and the conductive thread 140 may include all of the fibers fabricated to have low electric resistance by using a metal, a metal oxide, a carbon-based conductive material, or the like.

For example, a fiber formed of a carbon nanotube (CNT) and a metal nano material is light and has excellent electro-conductive and mechanical properties, so that the fiber is utilized as a conductive thread material transmitting various sensing information, and may include an application range of an electrode material storing energy. Various sensing information may be wired or wirelessly transmitted to the mobile device 200, which is in contact with or in proximity to the garment.

In an embodiment, the fabric device 100 may include the controller 130 sensing a result of the sensing of the sensor 110. The controller 130 and the sensor 110 may be electrically connected to each other through the conductive thread 140.

The controller 130 may generate a plurality of different signals, for example, first and second signals, according to a result of the sensing of the sensor 110. Further, the generated first and second signals may be provided to, for example, the mobile device 200 by using wired or wireless communication. In some embodiments, the controller 130 may also be omitted as necessary. When the controller 130 is omitted from the fabric device 100, and the controller 230 of the mobile device 200 is shared, the fabric device 100 may be configured to be well resistant to washing.

In some embodiments of the present invention, the fabric device 100 may include a garment. In this case, it is possible to discriminate whether the user wears the garment, the kind of worn garment, authenticate the user for the garment, whether the user wears a plurality of garments, whether the mobile device 200 is proximate to or in contact with the sensor 110, or the like by using the sensing information of the sensor 110 mounted inside/outside the garment.

In an embodiment, only when user authentication information of the garment is matched with user authentication information of the mobile device 200, it is possible to set the sensor information of the garment to be exchangeable. That is, when the user authentication information is matched, the mobile device 200 is operated in a first mode and accesses the sensing area 120 included in the garment, but when the user authentication information is not matched, the mobile device 200 is operated in a second mode and may not access the sensing area 120 included in the garment.

In this case, the user authentication may include all of fingerprint authentication, authentication utilizing biometric information, or the like.

Further, as one exemplary embodiment, it is possible to sense whether the conductive thread 140 of the garment is proximate to or in contact with a specific configuration of the mobile device 200. Accordingly, it is possible to recognize state information of the mobile device 200 incorporated in the garment. For example, it is possible to discriminate “a case where a display-touch interface 210 of the mobile device 200 faces inside an overcoat” from “a case where the display-touch interface 210 of the mobile device 200 faces outside the overcoat” by sensing direction information from the mobile device 200 incorporated in an inner pocket of the overcoat.

The direction information may include rotation direction information (yaw, pitch, roll, or the like) for the conductive thread 140 of the garment based on the display-touch interface 210. It is possible to differentially provide movement or touch sensing information, biometric information, output information, or the like through the garment based on the discriminated direction information of the mobile device 200.

In an embodiment, it is possible to recognize movement information of the wearer of the garment through the sensor 110 of the garment. A location or kind of the sensor to be mounted in the garment may be determined according to a body region where the user wears the garment and a main physical parameter (bending, heat, expansion/shrinkage, heartbeat, breath, or the like) generated in the body region.

For example, the movement information may be recognized or estimated through an image sensor, a geomagnetic sensor, an acceleration sensor, a proximity sensor, a breathing sensor, an ECG sensor, a PPG sensor, an EEG sensor, a pulse sensor, or the like. The sensed movement information may be transmitted to the mobile device 200, which is in contact with or in proximity to the garment, through the conductive thread 140 which can be utilized as trigger information for various control commands.

Further, the fabric device 100 and the mobile device 200 may include a communication means transceiving information with an external device. Here, the communication means may include a wired or wireless communication standard. In an embodiment, the communication means may include a communication channel for negotiating a voltage, a current, a charging direction, and the like through a wired cable. Further, the communication means may also include a communication interface, which is the wireless charging standard.

However, the fabric device 100 may also use various communication means within the mobile device 200. For example, as illustrated in the FIG. 2, in an embodiment, in which a controller 430 is shared with the mobile device 200, various information sensed by the sensor 310 may be transmitted by using the communication means within the mobile device 200. In this case, the fabric device may be implemented only with the sensor using a conductive thread 340 of the garment. Further, as long as the fabric device 100 is separated from a mobile device 200, the fabric device 100 may be conveniently and easily washed.

The fabric device 100 may include a smart belt, a smart bag, smart shoes, and a smart hat, in addition to the garment. More particularly, the fabric device 100 and a fabric device 300 may be configured as a wearable device, which surrounds at least a part (a wrist, a foot, a shoulder, a waist, a face, a head, or the like) of the body of the user with a plurality of surfaces when being used.

The mobile devices 200 and 400 may include, for example, a smart watch, smart glasses, a portable battery, a communicator, or the like. More particularly, the mobile devices 200 and 400 may be configured as a wearable device, which surrounds a specific portion (a wrist, a foot, a shoulder, a waist, a face, a head, or the like) of the body of the user. In other embodiment, the mobile devices 200 and 400 may be a smart phone, a personal digital assistant (PDA) or the like. The mobile device 200 may include an input unit receiving a user input (for example, all types of input such as a touch input, a gesture input, a voice input, or the like) related to wireless charging.

Hardware or software embedded a hardware means such as a controller (CPU), a battery, a display, a wired or wireless communication unit, and a sensor unit, of the mobile device 200, which is in the state of being in contact with or in proximity to the fabric device 100 and is electromagnetically connected, may be utilized to transmit/confirm/control various sensing information sensed by the garment.

The mobile device 200 may include the display-touch interface 210, a processing unit 220, and the controller 230.

The display-touch interface 210 may be disposed on, for example, a surface of at least a part of the mobile device 200. In an embodiment, the display-touch interface 210 may receive a touch input provided to the sensing area 120. In this case, the display-touch interface 210 and the sensing area 120 may be combined so that configurations of interfaces thereof correspond to each other, or are different from each other.

The processing unit 220 may perform a calculation required for driving the mobile device 200. In an embodiment, the processing unit 220 may include a processor and a storage unit, in which a program code required for processing the processor is stored. A result of calculating the program code through the processor may also be output through the display-touch interface 210. For example, when the processing unit 220 is driven with a first program code, the display-touch interface 210 may provide a first interface, and when the processing unit 220 is driven with a second program code different from the first program code, the display-touch interface 210 may provide a second interface different from the first interface.

The controller 230 may receive the first and second signals output from the controller 130 to control an operation of the processing unit 220 or the display-touch interface 210. That is, when the first signal is provided from the controller 130, the controller 230 may control the processing unit 220 or the display-touch interface 210 so that the mobile device 200 is operated in the first mode. Similarly, when the second signal is provided from the controller 130, the controller 230 may control the processing unit 220 or the display-touch interface 210 so that the mobile device 200 is operated in the second mode.

For example, when the controller 230 receives the first signal from the controller 130, the controller 230 may determine that the user of the fabric device 100 is in a first state, and be operated in the first mode. Similarly, when the controller 230 receives the second signal from the controller 130, the controller 230 may determine that the user of the fabric device 100 is in a second state different from the first state, and be operated in the second mode different from the first mode.

In an embodiment, when the controller 230 receives the first signal from the controller 130, the controller 230 may determine that the user of the fabric device 100 takes a first action, and be operated in the first mode. Similarly, when the controller 230 receives the second signal from the controller 130, the controller 230 may determine that the user of the fabric device 100 takes a second action, and be operated in the second mode.

Some non-limiting examples of the first action and the second action is represented in Table 1 below, but the technical spirit of the present invention is not limited thereto.

TABLE 1

| First action | Second action |
|---|---|
| Sleep | Wake |
| Move | Stop |
| Stand | Sit |
| Exercise | Break |
| Eat | Rest |

The smart interaction device 1 may adaptively change the operation mode (for example, the first mode and the second mode) of the mobile device 200 according to the state of the user of the fabric device 100. Here, the change of the operation mode may include a change of an output interface of the mobile device 200, a change of an application executed in the mobile device 200, an on/off change of a power supply of the mobile device 200, or the like.

FIG. 2 is a block diagram of a smart interaction device 2, according to the embodiments disclosed herein. In an embodiment, the smart interaction device 2 may include the fabric device 300 and the mobile device 400. Hereinafter, differences from the aforementioned exemplary embodiment will be mainly described.

A conductive thread 340, a non-conductive thread 350, a sensor 310, and a sensing area 320 included in the fabric device 300 may be substantially the same as those of the aforementioned fabric device 100 of the FIG. 1. However, the fabric device 300 may not include the controller 130 of the FIG. 1.

In an embodiment, the function of the controller 130 of the FIG. 1 may be performed by a controller 430 of the mobile device 400. That is, the controller 430 of the mobile device 400 may sense sensing results of the sensor 310 and a sensing area 320 disposed in the fabric device 300 by using the conductive thread 340 included in the fabric device 300, and control an operation of a processing unit 420 or a display-touch interface 410 based on the sensing results.

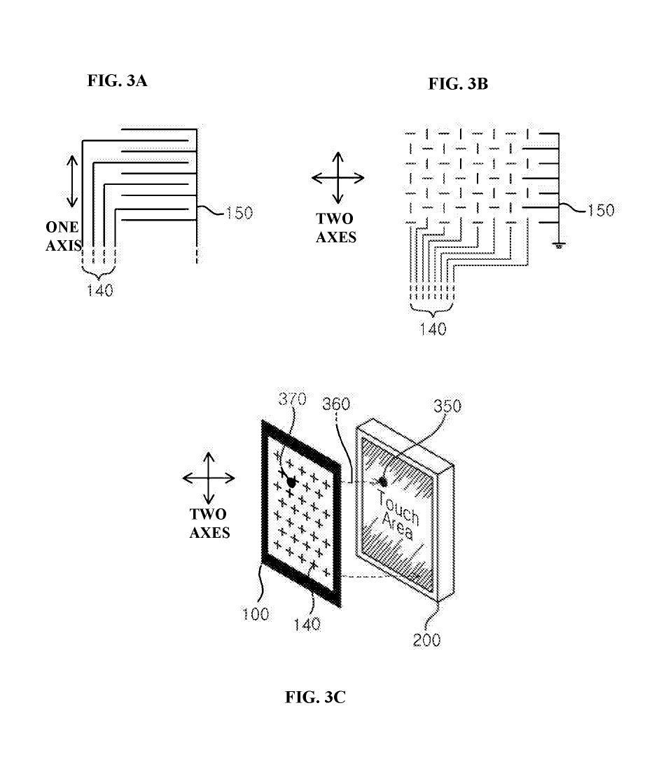

FIGS. 3A to 3C are diagrams illustrating characteristics of a touch input according to a conductive thread pattern and a touch reaction to a display of an external device electromagnetically connected with a conductive thread, according to the embodiments as disclosed herein.

The FIG. 3A illustrates an example, in which a conductive material and a non-conductive material stitched together by crossing each other and thus a touch input signal for one axis is generated. Consequently, non-conductive thread is intervened between two conductive threads and prevent them from being electrically connected. The touch input signal of one axis by the stitch method may be transmitted to an external device (for example, the mobile device 200 of the FIG. 1) electromagnetically connected with the fabric device to generate various touch input command signals.

In the FIG. 3B, a conductive thread pattern is configured in a different pattern from that of the FIG. 3A, so that a touch input signal of two axes may be generated. The generated touch input signal may be transmitted to the external device electromagnetically connected with the fabric device. The pattern of the conductive thread 140 formed by various stitch methods as illustrated in the FIGS. 3A and 3B may generate various touch input command signals in spite of the same touch.

FIG. 3C is a diagram illustrating a touch reaction characteristic of the mobile device 200 electromagnetically connected with the fabric device through a conductive surface. The conductive surface is an area formed of various stitch patterns of the conductive thread, and becomes a virtual touch area (touch-through area). The virtual touch area described herein is correspond to at least a part of the touch area of the mobile device 200 positioned inside a pocket of the garment. For example, the pattern provided within the fabric device 100 is electromagnetically connected with the external device so that a case where a touch pattern 150 of the fabric device 100 exhibits the same effect as that of a case where a specific area of the touch area of the mobile device 200 is touched. That is, it is possible to sense a reaction to the touch based on a change in capacitance or a resistance signal in the display of the external device, which is electromagnetically connected with the conductive surface, by the touch to the conductive surface of the fabric device.

In this case, a touch input to the virtual touch area (touch-through area) of the conductive surface may mimic one-to-one and can be recognized by the display of the external device. The touch input signal recognized by the external device is changed according to a state (an area, a conductive pattern, a material, a density, or the like) of the virtual touch area, a physical (size, weight, or the like) or electromagnetic (sensitivity, a sensing method, or the like) condition of the display of the external device, the kind or function of executed application of the external device when a touch is input into the virtual touch area, the kind of garment, or the like, A touch command signal may be appropriately encoded in response to the touch input signal and be transmitted to a processor of the external device.

For example, a specific point 370 within the touch pattern 150 of the fabric device 100 is configured to correspond to a specific point 350 of a touch screen of the mobile device 200. Further, when the user touches the specific point 370, the specific point 350 of the touch screen is touched through mimicking 360. For example, the specific point 350 of the touch screen may be a back button of a web page of a mobile browser implemented with a graphic user interface. That is, when the specific point 370 within the touch pattern 150 of the fabric device is touched, the web browser of the mobile device 200 may perform the control of returning to a previous page.

Otherwise, the specific point 350 of the touch screen may be a play button of a music player implemented with the graphic user interface. That is, when the user touches the specific point 370, the fabric device 100 may also be implemented so that the mobile device 200 plays music. In addition, it is possible to achieve an effect, such as a touch of various buttons of the graphic user interface, through the implementation of FIG. 3C.

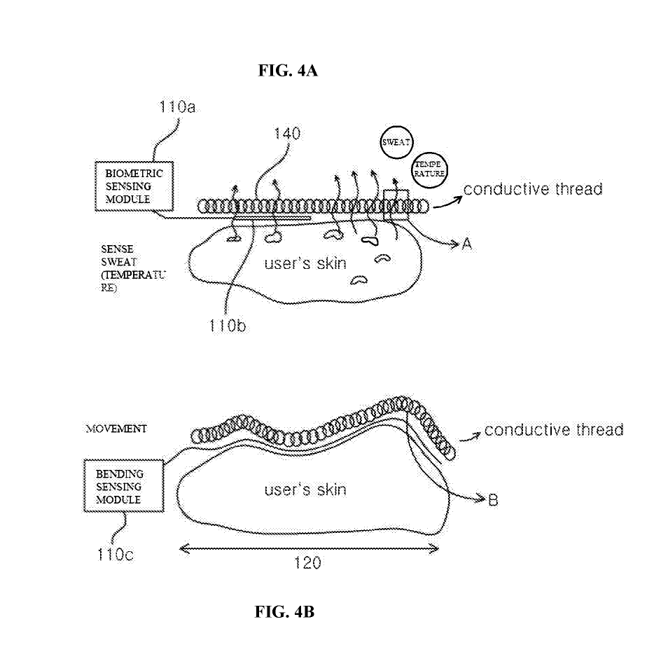

FIGS. 4A and 4B are diagrams illustrating a characteristic of sensing information of the user by using the conductive thread in the sensing area 120, which is in contact with or in proximity to skin, according to the embodiments as disclosed herein.

Referring to the FIG. 4A, the sensing area 120 of the FIG. 1 may include a biometric sensing module 110a. The biometric sensing module 110a may sense a physical or chemical change according to heating or sweating of the user. The biometric sensing module 110a may transmit the sensed change to the mobile device 200 through the conductive thread 140 connected with a sensor electrode 110b.

Referring to the FIG. 4B, the sensing area 120 of the FIG. 1 may include a bending sensing module 110c. The bending sensing module 110c may transmit information sensed according to a movement of the wearer to an external device at another location through the conductive thread 140. Further, the sensing area 120 including a partial area of the conductive thread 140 may transmit information sensed according to an electric resistance change of the conductive thread 140 to the mobile device 200 through the conductive thread 140.

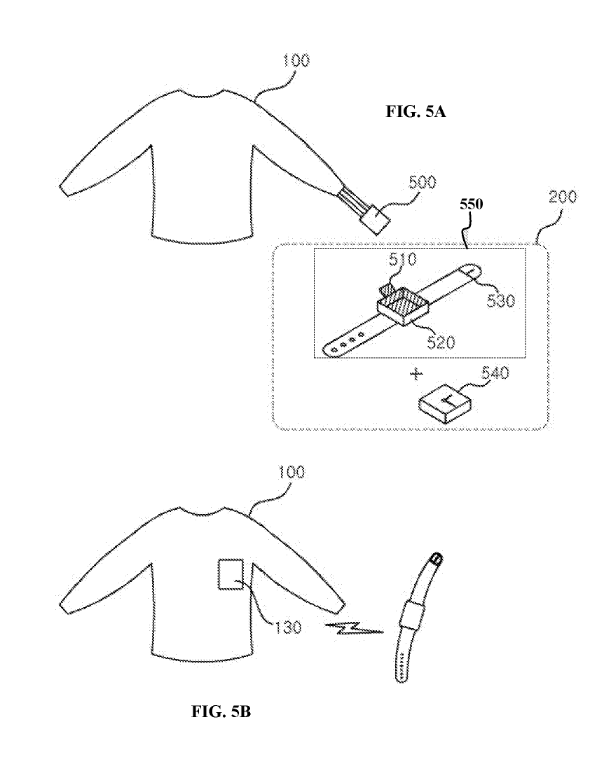

FIGS. 5A and 5B are diagrams illustrating a form of an interface, through which the mobile device 200 and the fabric device 100 may transmit and receive a signal, according to the embodiments as disclosed herein.

Referring to the FIG. 5A, the fabric device 100 is connected with the mobile device 200 through a connector 500. According to an embodiment of the present invention, the fabric device 100 is connected with the mobile device 200 using a wired connection. The connector 500 is connected with a plurality of signal lines of the fabric device 100.

The mobile device 200 includes a main body 540 and an interface 550 which includes a case 520, a strap 530, and a connector 510. The connector 510 is a configuration, which may be detachably coupled to the connector 500, and the case 520 is configured to accommodate the main body 540.

However, the connector 500 may adopt a configuration having other scheme, other than the standard communication scheme. For example, when a touch sensing area is included in the fabric device, a plurality of touch sensing lines may be connected to the connectors 500 and 510 as it is. In this case, the case 520 may include a control module capable of receiving a signal from the touch sensing line and processing the received signal, and a sensing unit capable of measuring a capacitance or resistance change of the touch sensing line. Further, the case 520 and the main body 540 may be connected by various wired or wireless communication schemes. In the present exemplary embodiment, the fabric device 100 does not require a separate communication function for the communication with the mobile device 200, so that the fabric device 100 may have a configuration resistant to washing.

The FIG. 5B illustrates a configuration, which does not separately include a separate case 520. The controller 130 includes a wireless communication device, and the controller and the mobile device 200 may be connected through wireless communication.

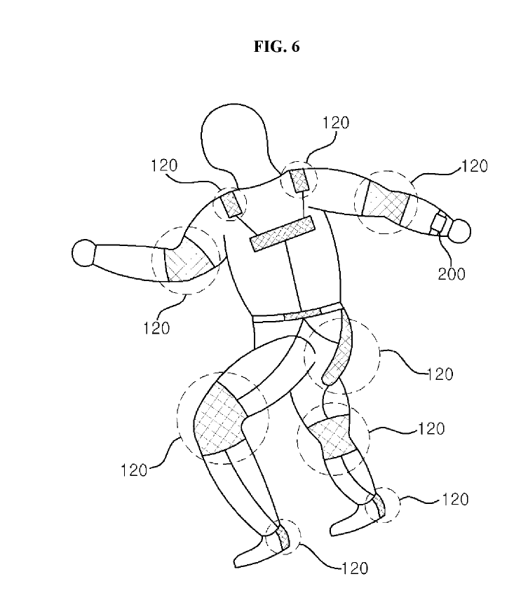

FIG. 6 is a diagram illustrating contents of sensing a pattern change in movements of a wearer by the garment to switch an operation mode of a smart watch, according to the embodiments as disclosed herein. Contents of locating the sensing area 120 to a region (for example, a joint region) having lots of motions of the body in order to sense movements of the user wearing the fabric device 100 is disclosed. As illustrated in the FIG. 6, the plurality of sensing areas 120 may be disposed in movable body regions (for example, joint regions) in the garment. The sensing information of the plurality of sensing areas 120 may be transmitted to the mobile device 200, which is in contact with or in proximity to the garment.

The mobile device 200, which is in contact with or in proximity to the garment and receives the sensing information, may determine an inflection point of an action of the user wearing the garment. The inflection point may mean a time point, at which a change in an action is most maximized and found. It is possible to determine an operation mode, an input/output mode, or the like of the mobile device 200 through information on the inflection point of the action of the user wearing the garment.

For example, the fabric device may sense an inflection point of an action that the wearer walks and then starts to run, and control so that a healthcare application of the mobile device 200 may be automatically executed based on the inflection time point or a voice input may be frequently received by continuously activating a microphone during the running.

In an embodiment, when an action of the user in a specific place is less, for example, in a case where an action of the user is less around a lecture room, the fabric device may determine the case as a lecture mode. Further, fabric device changes an alarm sound to mute and execute a sleep mode. In an embodiment, the fabric device may control the mobile device 200 to be operated in a charging mode by supplying power to the mobile device 200 from another portable device connected with the fabric device 100.

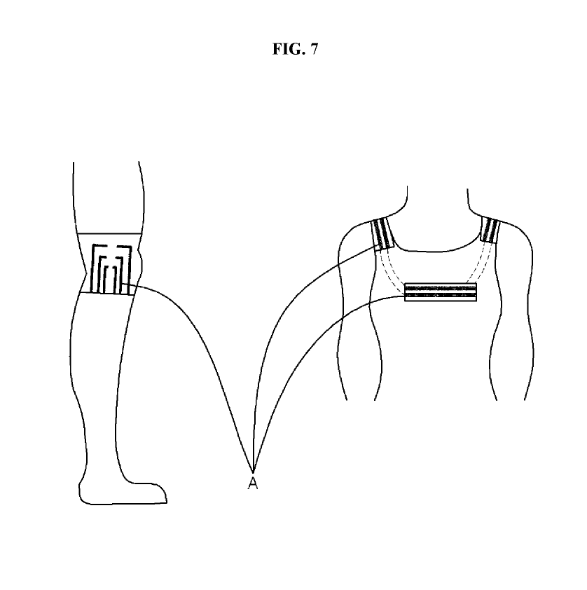

FIG. 7 is a diagram illustrating contents of providing a conductive thread to a position, at which a joint region is bonded, in order to sense a movement of a wearer of the garment, according to the embodiments as disclosed herein. In an embodiment, the FIG. 7 illustrates a state where a conductive thread (textile sensing band) A having elasticity, which is provided in a predetermined region of the garment in order to sense the movements of the wearer according. Particularly, the conductive thread A having elasticity may be positioned in a body region such as a knee, a shoulder, a chest, or the like. Sensing information sensed through the conductive thread A having elasticity may be transmitted to the mobile device 200 so that the mobile device 200 may sense an inflection point of an action of the wearer of the garment.

In an embodiment, a pattern, an area, density, or the like of a conductive thread formed of the conductive thread A may be variously changed according to a characteristic of a body region. As an example, when the wearer sits for a long time and then stands up, an operation mode of a smart watch B of the FIG. 6 or the kind of application to be executed may be determined based on an inflection point of the action of the wearer of the garment based on a resistance (current) change at a hip and a knee.

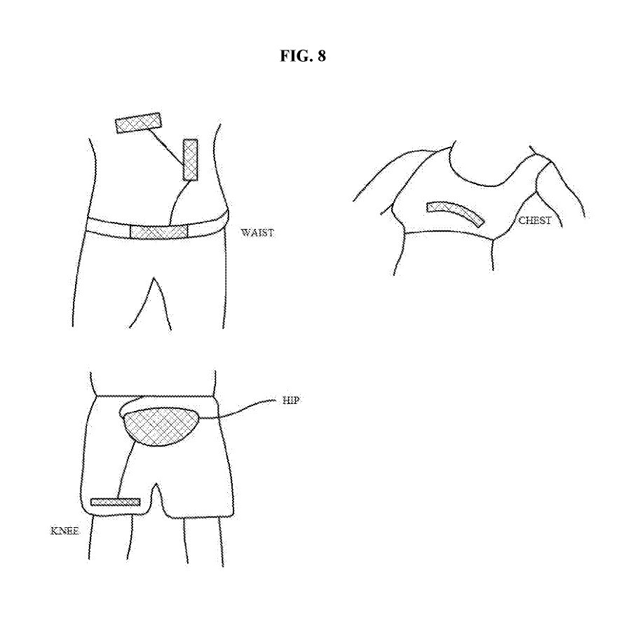

FIG. 8 is a diagram illustrating a conductive thread having elasticity, which is provided in a predetermined region of a garment in order to sense a movement of a wearer, according to the embodiments as disclosed herein. In an embodiment, the FIG. 8 illustrates example contents for a position characteristic of a conductive thread in order to effectively sense an inflection point of the movements of the wearer of the garment. The conductive thread may be formed in a region of the garment corresponding to a body region, in which an inflection point of the movements is sensed well, and a stitch method of the conductive thread may be changed according to a characteristic of the body region.

In an embodiment, it is possible to sense a touch input of the wearer of the garment through the conductive thread. A touch-through input is a touch to a display of an external device, which is in contact with or in proximity to the conductive thread. The sensed touch input is utilized as control command information for the external device which is in contact with or in proximity to the conductive thread.

Further, when a plurality of devices is mounted in the garment, a touch-in distance input is also allowed. That is, a touch input to a first device may be recognized as control command information for a second device mounted at another position, which is not in contact with or in proximity to the conductive thread. Whether the touch input through the conductive thread is recognized as the touch-through input or the touch-in distance input may be determined by the kind of garment, the number of worn garments/devices, a touch input method, or the like.

The garment may have different virtual touch input information recognized according to the kind of conductive/non-conductive material, a crossing method, and a pattern according to various stitches. The garment may recognize a contact touch area for the conductive thread through a display of the external device, and recognize an external input to the garment as a touch input to the display of the external device based on a conductive thread pattern and contents of the conductive thread within the touch area, in addition to the recognized touch area. In addition, it is possible to correct the touch input by considering a difference in a normal line vector between the garment formed of the conductive thread and the display of the external device. In this case, a correction value of the touch input may be determined by additionally considering a stitch method of a conductive surface, a material of a conductive surface, a relative difference in the normal line vector according to a position of a conductive surface formed in the garment, or the like.

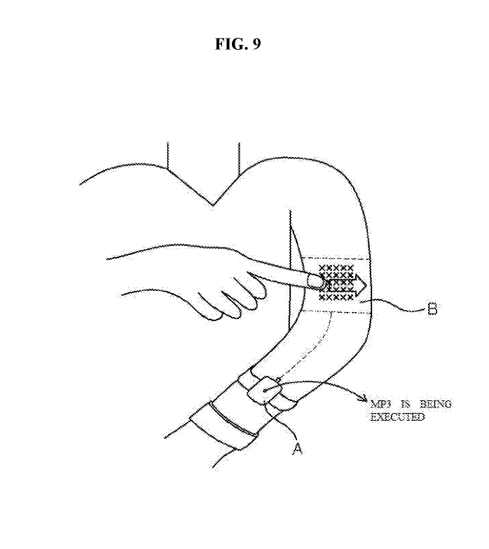

FIG. 9 is a diagram illustrating an example scenario in which a touch input to an external device which is in contact with or in proximity to a garment, according to the embodiments as disclosed herein. In an embodiment, a characteristic of controlling an external device connected with the conductive thread at another position through a virtual touch input to a conductive surface of the garment is described in the FIG. 9. A display A of the external device may be in contact with a virtual touch area B formed of a conductive thread to control the external device.

In an example embodiment, it is possible to operate an MP3 player, which is being executed in the external device.

In another example embodiment, it is possible to perform a scroll function (next music, previous music, volume up and down, or the like) of the display A of the external device by sensing a pattern of a change in a touch resistance value by a conductive thread pattern configuration of the virtual touch area B.

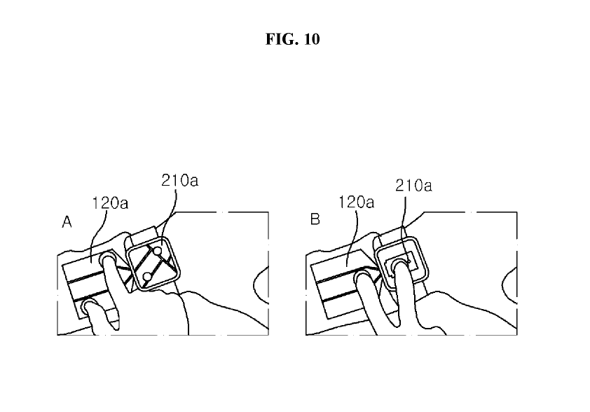

FIG. 10 is a diagram illustrating a virtual touch area of a sleeve of a garment and a touch input to a display of a smart watch, in a state where a connection terminal of the sleeve of the garment is physically or electromagnetically connected with a connection terminal of the smart watch, according to the embodiments as disclosed herein. In an embodiment, the FIG. 10 illustrates touch recognition characteristics of the garment and the smart watch, which is in contact with or in proximity to the garment. A is an example embodiment, in which a sleeve of the garment electromagnetically connected by an external device, which is in contact with or in proximity to the garment, is utilized as a virtual touch area 120a.

According to the kind or a function of executed application of the smart watch, contents are displayed on the smart watch, and a command for the smart watch may be performed by various touches (multi-touch/single touch) to the virtual touch area 120a. As described above, it is possible to input a generally and commonly used touch gesture such as a scroll movement, a multi-tasking selection, a screen rotation, or the like by utilizing two fingers into the external device (for example, the smart watch) based on a directional input of an electrical resistance change for the virtual touch area of the sleeve.

In an embodiment, the touch input may be performed by variously combining the multiple touches of a first touch and a second touch with the virtual touch area 120a of the sleeve and a display area 210a of the smart watch. For example, when a map application is displayed and executed on the display area 210a of the smart watch, a zoom-in and zoom-out function may be executed by touching each of the display area 210a of the smart watch and the virtual touch area 120a of the sleeve of the garment.

Further, a differential command may be performed through sequential touch inputs to the display area 210a and the virtual touch area 120a.

In an embodiment, the garment may recognize biometric information of the wearer of the garment through a mounted internal/external sensor. The garment may recognize information such as sweat, pulse, breathing, a temperature, pulse, brain waves, blood pressure, oxygen saturation, or the like of the wearer of the garment through the biometric sensor (such as the ECG sensor, the PPG sensor, the EEG sensor, the pulse sensor, the breathing sensor, the SPO2 sensor, the blood pressure sensor, the electroencephalogram sensor, or the like). In this case, the garment may recognize the biometric information of the wearer of the garment through a physical parameter change sensed by the conductive thread. The biometric information sensed by the sensor or the conductive thread is transmitted to the contact/proximate external device through the conductive thread.

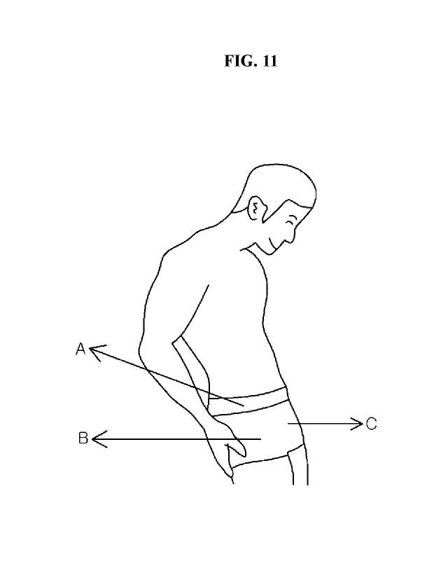

FIG. 11 is a diagram illustrating characteristics of various body movements and biometric information detected by an underwear, according to the embodiments disclosed herein. In an embodiment, the FIG. 11 illustrates contents of sensing body/physiological information, movement information, or the like about a user through various biometric sensors (for example, reference numeral 110 of the FIG. 1) provided in the garment such as an underwear. A type, structure, or the like of the biometric sensor is changed according to a position characteristics of positions A, B, and C, as shown in the FIG. 11.

In an embodiment, it is possible to obtain body information and biometric information, such as blood pulse and a heart rate, of a user through a biometric sensor provided at the position A. It is possible to sensitively measure a body (for example, a lower body) movement of the user, and sense various movement information such as a lower body exercise, walking, running, and bending through a sensor provided at the position B. A sensor provided at the position C may sense various physiological information about the user or the like.

In an embodiment, it is possible to output movement or touch recognition information and biometric recognition information sensed by the sensor or the conductive thread of the garment through various output means. Further, control commands for the garment and the external device may be generated based on the sensing information.

When the output information is provided, it is possible to utilize an output means of the external device, which is in contact with or in proximity to the garment. Various sensing information of the garment may be provided in various modality forms of the output means by utilizing the output means, which is capable of providing five senses including sense of vision, sense of hearing, sense of touch, or the like of the external device.

It is possible to provide the user with various sensing information of the garment by controlling a physical or chemical parameter such as transparency, a color, a distortion, a temperature, a density, or the like of the conductive thread. In an embodiment, it is possible to control the conductive thread based on the sensing information.

In an embodiment, when a heating state having a predetermined level or more is sensed through the sensor of the garment, the garment may enable the user to recognize the biometric state by adjusting transparency, a color, or the like of the conductive thread. Simultaneously, the garment contribute to decrease a degree of heating by automatically or passively adjusting the temperature, the density, or the like of the conductive thread.

Further, it is possible to provide the output means with power of the external device, which is in contact with or in proximity to the garment, through a wired or wireless power transmission method. Otherwise, the conductive thread formed of a carbon nano tube or metal nano material formed of an electrode material may provide the output means with power, which is stored by the conductive thread itself. That is, the garment and the external device may exchange electrical energy with each other by using the conductive thread.

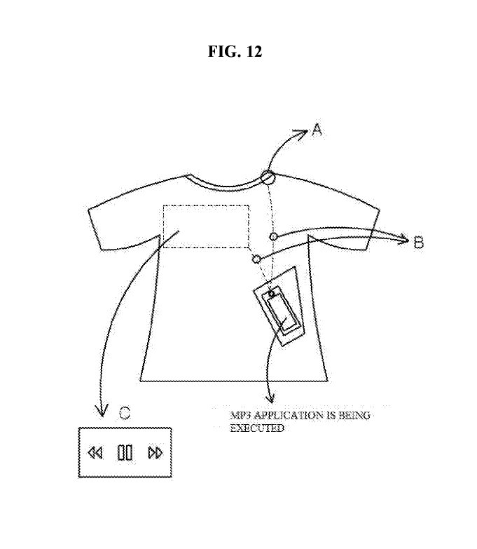

FIG. 12 illustrates a characteristic of transmitting voice information reproduced by an external device to a wearer through a bone conduction speaker provided at a collar of the garment, according to the embodiments as disclosed herein. In an embodiment, a characteristic of transmitting and outputting reproduction information of an external device, which is in contact with or in proximity to a garment, to a bone conduction speaker proximate to the ears are described.

A sound source of an MP3 is transmitted to a collar A close to the ear of the user through a conductive thread B so that the user may listen to music through bone conduction of skin coming into contact with the collar.

Further, various electrical signals of the garment may be transmitted to the contact, or proximate worn or incorporated external device through the conductive thread B. The various received electrical signals may be encoded as command signals related to a currently executed application or function.

A screen display user interface (UI) C may be changed in response to the encoded command signal. In this case, a touch interface of the external device (for example, the MP3) may include a first area and a second area. The screen display UI C may include a third area physically corresponding to the first area and a fourth area physically corresponding to the second area.

Here, a touch input sensed in the third area may be recognized as an input to the first area of the touch interface, and a touch input sensed in the fourth area may be recognized as an input to the second area of the touch interface.

Further, an output of the external device (for example, the MP3) receiving the touch input sensed in the third area and the touch input sensed in the fourth area of the screen display UI C may be transmitted to the collar A close to the ear of the user through the conductive thread B. The user may listen to music through bone conduction.

A terminal of the garment is in contact with and electromagnetically connected with a terminal of the external device, and touch/gesture information, posture/movement information, and biometric information sensed by various sensors of the garment may be transmitted to the external device through the conductive thread in the connection state.

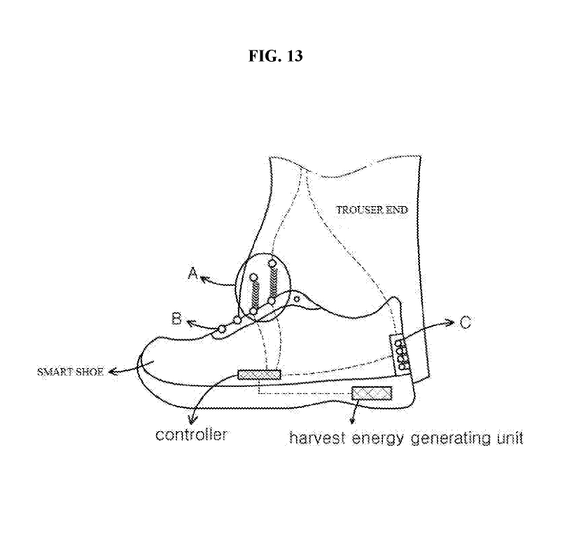

FIG. 13 is a diagram illustrating a characteristic of transceiving sensing information with a shoe which is in contact with or in proximity to a garment, according to the embodiments as disclosed herein. In an embodiment, as illustrated in region A, a connection terminal of a shoe strap may be in contact with a connection terminal of a trouser end to connect upper and lower garment with the smart shoe. As illustrated in region B, a shoe strap itself may be in a form of a cable battery or a conductive thread. As illustrated in region C, a connection terminal of a heel of the smart shoe and a connection terminal inside the trouser end may be coupled to each other by a contact means. Movement information and physiological information of the garment, step information, posture information, foot health information of the smart shoe, or the like may be processed by an external device, which is in contact with and connected to another position of the garment.

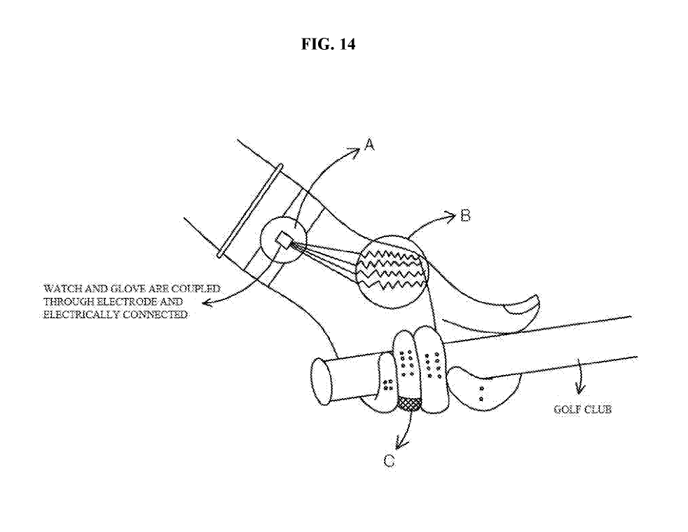

FIG. 14 is a diagram illustrating a characteristic of transceiving sensing information with a glove which is in contact with or in proximity to a garment, according to the embodiments as disclosed herein. In an embodiment, information sensed by a watch A (or the glove) may be received through visual, audible, and touch means.

Grip intensity of a sport tool, sweat quantity, a rotation degree, or the like may be sensed through a sensor B provided in the glove and the sensing information may be transmitted to the connected smart watch.

A finger movement within the glove may be sensed by a sensor C provided in the glove, and a sensed finger movement signal is transmitted to the connected smart watch through a conductive thread provided in the glove. The received finger movement signal is encoded into command information corresponding to the currently executed application or function, and performs various commands.

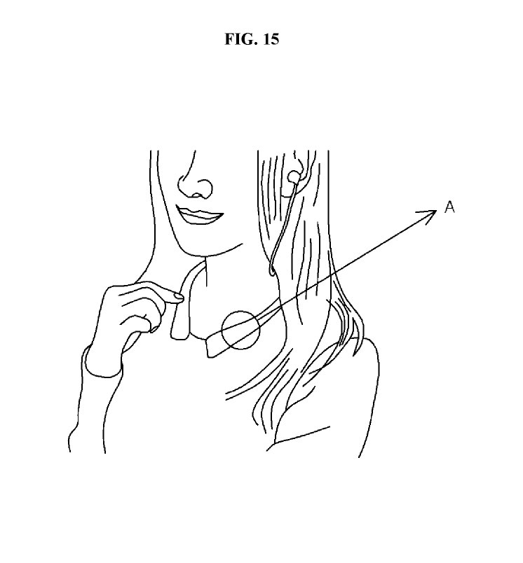

FIG. 15 is a diagram illustrating a characteristic of transceiving information between a garment and a wearable device which is in contact with or in proximity to the garment, according to the embodiments as disclosed herein.

In an embodiment, as illustrated in region A, a predetermined region of the garment may be in contact with and electrically connected with a headset. In this case, power may be exchanged between the garment and the headset through a wireless charging coil in the predetermined contact region. A function of the headset may be controlled with a virtual touch area of the electrically connected garment.

In an example, 1) whether the garment is in contact with the headset or is normally connected with the headset, 2) wireless charging related information (a progressing state, a contact guide, and charging feedback), 3) interaction related information for controlling the headset through the garment (a control command performance result and interaction guide), or the like may be output to a user in an audio form through an earphone connected with the headset or bone conduction.

In another example, when an earphone of the headset is detachable in a wireless form, in addition to a wired form, it is possible to guide whether the earphone may be attached or detached by considering a charging state of the earphone during the progress of wirelessly charging the garment.

Further, since it is difficult to control a touch of the headset worn on the neck of a user, when various events (a notification, a control, or the like) related to an executed application of the headset are generated, a physical signal according to a touch or a movement sensed in a predetermined region of the smart garment may be received through a control UI of the headset.

In this case, a function of a command input according to the physical signal may correspond to a function required by the event related to the executed application. Further, when a control input related to the executed application of the headset is required, it is possible to guide that a control input related to the headset may be performed through a specific region by providing visual, audible, and touch feedback to the specific region of the garment connected with the headset.

When a plurality of garments formed of the conductive thread is worn and overlaps, the plurality of garments may be electromagnetically connected with each other through the overlapping conductive surfaces and areas. Further, when the overlapping wearing is sensed through the sensors of the garment, it is possible to set an external device, which is worn or incorporated while being in contact with or in proximity to one of the plurality of garments, as a device controlling the plurality of garments. In this case, even when the recognized touch input information is changed by the overlapping of the plurality of garments, it is possible to set the different touch inputs to be recognized as the same input.

The plurality of worn or incorporated external devices, which is in contact with or in proximity to the garment, may transceive various sensor information through the conductive thread. The plurality of external devices may have one operation mechanism by the electromagnetically connected garment, and sensing information of the garment may be processed by a part of the plurality of external devices. The plurality of external devices may be divided into a master or slave device, and differentially process the sensing information of the garment. In an embodiment, the master device may convey a command to the slave device.

The plurality of garments may be in contact with and electromagnetically connected with the plurality of external devices, respectively. A touch-through input to an electromagnetically connected first external device may be processed as a command (touch in-distance input) for a second external device, which is in contact with another position of the garment. In an embodiment, one of the plurality of garments may not be a general garment. For example, the garment may be a backpack, hat, shoes, bag or the like made of the conductive threads.

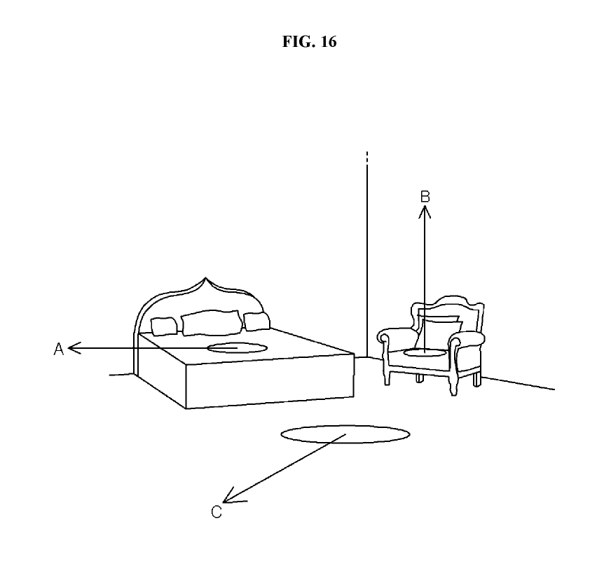

FIG. 16 is a diagram illustrating an example scenario in which contents of a contact is exchanged between a garment and an external object, according to the embodiments as disclosed herein.

In an embodiment, when surfaces of objects, such as a bed A, interior decoration, a floor, a chair, or the like are formed of a conductive thread or a conductive material, or are separately conductive-processed, the objects may be recognized as the garment of the present invention.

In an embodiment, when a user sits on the chair, a seat portion B of the chair is in contact with a hip portion of the worn garment. In this case, the user may be connected with an external device or a specific service related to the chair, as well as internal operation hardware and software provided in the chair. As one exemplary embodiment, a head mounted display (HMD) such as smart glasses, is in contact with or in proximity to the garment, so that the HMD may provide the electromagnetically connected garment with a function or feedback related to virtual reality (VR) information provided by the HMD. In this case, the HMD may provide more realistic contents immersion by providing a feedback stimulating five senses through cooperation with the external connected Internet of Things, other than the garment.

In an embodiment, when a user wears socks provided with the conductive thread and enters a specific space, data may be exchanged while the socks of the user may be in contact with the floor C of an indoor side, so that the user may be connected with a change in an inner side of the floor or an external device and a specific service. In this case, the garment may recognize a user's feeling and reproduce music for acclimating according to a temperature and a state of the feet.

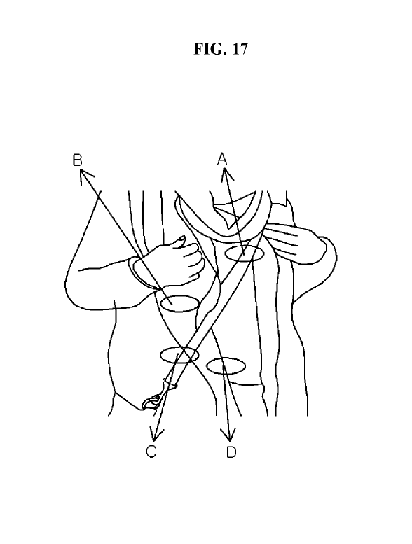

FIG. 17 is a diagram illustrating a characteristic of a contact and a control characteristic between a garment and an accessory, according to the embodiments as disclosed herein.

FIG. 17 illustrates a characteristic of a contact and a control characteristic between the garment and the accessory in the exemplary embodiment of the present invention. As show in the FIG. 17, Point A is a part at which the accessory formed of a conductive thread is in contact with a conductive surface (fabric side) of the garment, point B is a part at which two or more conductive surfaces of the garment are in contact with each other, point C is a part in which the conductive accessory is in contact with the conductive surface of the garment, and point D is a part at which two kinds of garment formed of a conductive surface are in contact with each other.

Recognition information of the plurality of devices connected with the plurality of garments may be utilized as a plurality of elements of device control information through the garment.

In the meantime, the garment may guide a posture or a movement of a user in order to sense required information in relation to an application executed in a worn/incorporated external device. Feedback information/means/energy or the like provided to the user may be changed according to the posture or movement information of the user required in the executed application.

Further, the garment may generate a command signal for the worn/incorporated external device electromagnetically connected with the garment by sensing a space gesture operation with a sensor provided at a specific position of the garment.

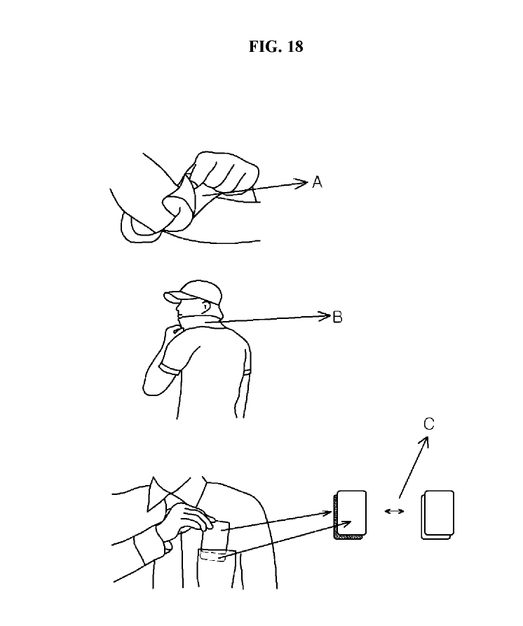

FIG. 18 is a diagram illustrating an interaction characteristic by various bonding methods between a specific region of a garment and an external device, according to the embodiments disclosed herein. In an embodiment, as shown in the FIG. 18, A illustrates an operation in which a resistance value of a sensor attached to the garment is changed by an operation of rolling up or spreading a sleeve. In this case, an electrical signal changed by the operation of rolling up or spreading the sleeve may be utilized as a command signal controlling a worn or incorporated external device electromagnetically connected with the garment.

As one exemplary embodiment, when a notification of a call or message is received in a mobile device incorporated in a pocket of the garment, the received notification may be processed by an operation for the sleeve.

B illustrates an example, in which an electrical signal change sensed by the sensor by a gesture of turning up a collar is utilized as a control command of an external device. In an embodiment, when a user turns on the collar, the electrical signal change may be utilized for switching a mode of a headset, which is in contact with the collar, from a speaker mode to an earphone mode or temporally stopping music contents provided from a Bluetooth earphone worn on the ears and switching a mode of a headset into a mode for receiving a call.

C illustrates a characteristic of sensing whether a predetermined region of a pocket formed of a conductive thread/sensor is in contact with or in proximity to a specific part of an external device, and controlling the external device. In an embodiment, when a display of the external device is proximate to or in contact with the conductive thread of the garment in a first direction, the proximity or contact may be recognized as a mode capable of receiving a control input such as a touch input (a touch-through input and a touch in-distance input), a space gesture input, and a voice input. When the display of the external device is in contact with or in proximity to the conductive thread of the garment in a second direction different from the first direction, the proximity or contact may be recognized as a mode in which it is impossible to perform a command by various control input means.

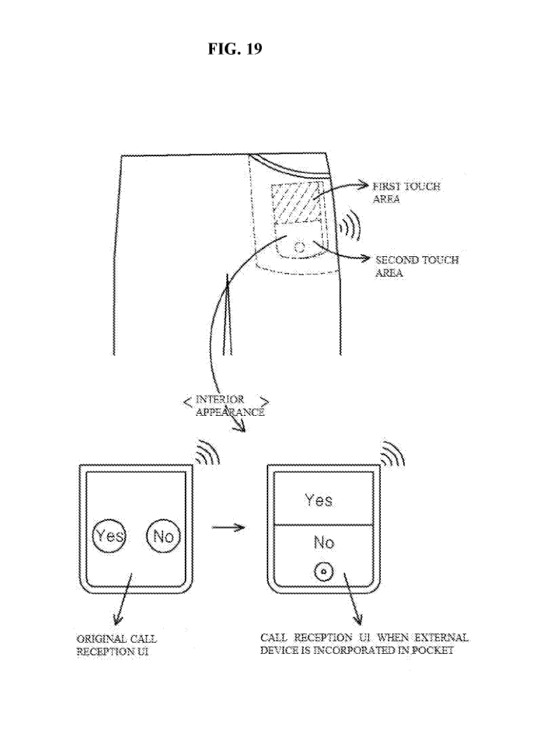

FIG. 19 is a diagram illustrating contents of the different configuration between a touch pattern input into an external device and a touch pattern input into a conductive thread with respect to the same event, according to the embodiments as disclosed herein.

In an embodiment, the FIG. 19 shows a characteristic of a control method of a real touch area of a display of an external device recognizing a touch (touch-through) input to a virtual touch area of a garment, which are in contact with or in proximity to the external device. A conductive surface of the garment, which is electromagnetically connected with the external device becomes a virtual touch area. A user may perform a touch input (touch-through) to the external device in a pocket of the garment, which are in contact with the virtual touch area through a touch input to the virtual touch area of the garment. In this case, the touch input to the virtual touch area may be one-to-one matched and be recognized by the display of the external device and a touch input environment such as a state (a conductive pattern, a material, density, or the like) of the virtual touch area, a physical information (size and weight) or electromagnetic condition (a communication means and sensitivity of the display) of the external device, the kind or function of executed application of the external device, and the kind of garment, may be defined.

The external device may encode a first touch input to the virtual touch area into a second touch input performed with a touch area, which is different from that of the first touch input, in response to the touch input environment, and recognize the first touch input as the second touch input. For example, as shown in the FIG. 19, when user puts the smart phone in pocket, the first UI is changed to second UI. When user touches upper side of second UI, the external device make the signal same to the signal made when the user press “Yes Button” of the first UI. Otherwise, a shape, a size, and a function of the real touch area of the display of the external device may be changed in response to the touch input environment. The real touch area of the display of the external device may be a GUI form.

That is, in several exemplary embodiments, the virtual touch area includes an area, which does not physically correspond to a touch interface of the external device, and a touch input sensed in the area may be recognized as an input to the touch interface of the external device.

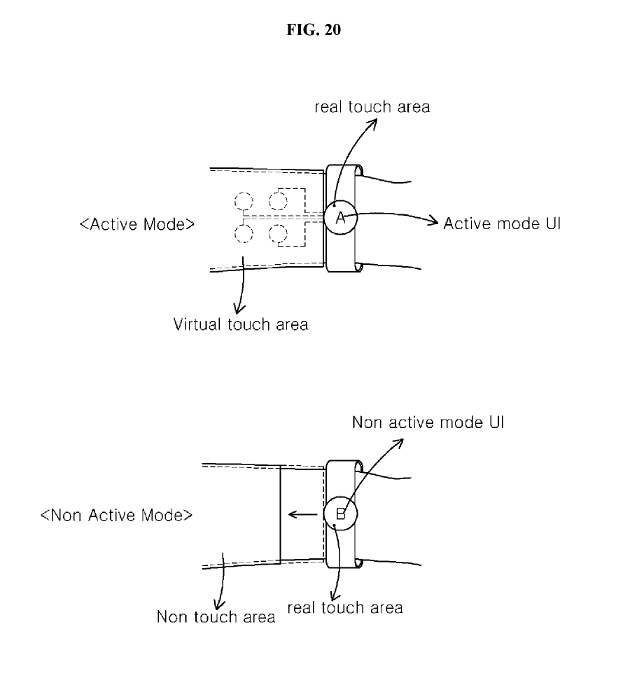

FIG. 20 is a diagram illustrating a characteristic of switching an operation mode of an external device in a state where a conductive thread of a garment is in contact with and electromagnetically connected with the worn external device, according to the embodiments as disclosed herein.

In an embodiment, a predetermined region of the garment is activated as a virtual touch area by the switched operation mode, and a touch input to the virtual touch area is recognized as an input of controlling an external device connected by the touch in-distance method. Further, a user interface, which is capable of manually setting how to process various electrical signals for the virtual touch area by a smart watch may be displayed.

In this case, even in a case of the same change in an electrical signal (resistance, heat, and input), a command recognized by the smart watch may be differently encoded according to the manual selection or the kind and a characteristic of executed application or function. Further, guide information for electrically connecting a terminal connected with the conductive thread and a terminal of the smart watch may be provided. Particularly, when an attempt to access is sensed through a proximity sensor around each contact terminal, the guide information may be provided.

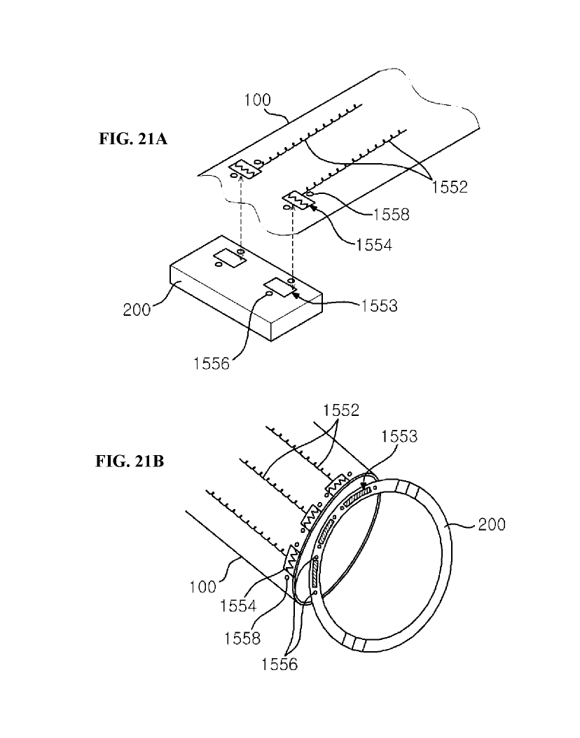

Hereinafter, various forms of connection methods between the mobile device 200 and the fabric device 100 will be described with reference to FIGS. 21A to 25.

FIG. 21A illustrates a connection between a connection terminal 1554 located within a sleeve of the fabric device 100 and a connection terminal 1553 of the mobile device 200. Referring to FIG. 21A, in an embodiment, the connection terminal 1553 of the mobile device 200 is located to be coupled with the connection terminal 1554 of the fabric device 100 at a location, at which the connection terminal 1553 of the mobile device 200 overlaps the connection terminal 1554 of the fabric device 100. In the embodiment, when the sleeve covers the smart watch, the smart watch is connected through the connection terminal 1554 of a first area provided in the fabric device 100. In this case, in order to easily connect or maintain a connection between the connection terminal 1554 of the sleeve of the fabric device 100 and the mobile device 120, the mobile device 120 may include an adhesion part 1556 formed of a magnet or an adhesive material. The adhesion part 1556 may provide a physical guide so that the fabric device 100 and the mobile device 120 are located at a position, at which the fabric device 100 and the mobile device 120 may be connected.

In an embodiment, an adhesion part 1558 may be provided at the sleeve of the fabric device 100 at a position corresponding to the adhesion part 1556 of the mobile device 120.

A structure of the FIG. 21B is basically the same as that of the FIG. 21A. However, in an embodiment, the connection terminals 1553 and 1554 and the adhesion parts 1556 and 1558 are configured so that the mobile device 120 is coupled to an end of the sleeve of the fabric device 100. Descriptions of the overlapping parts are omitted.

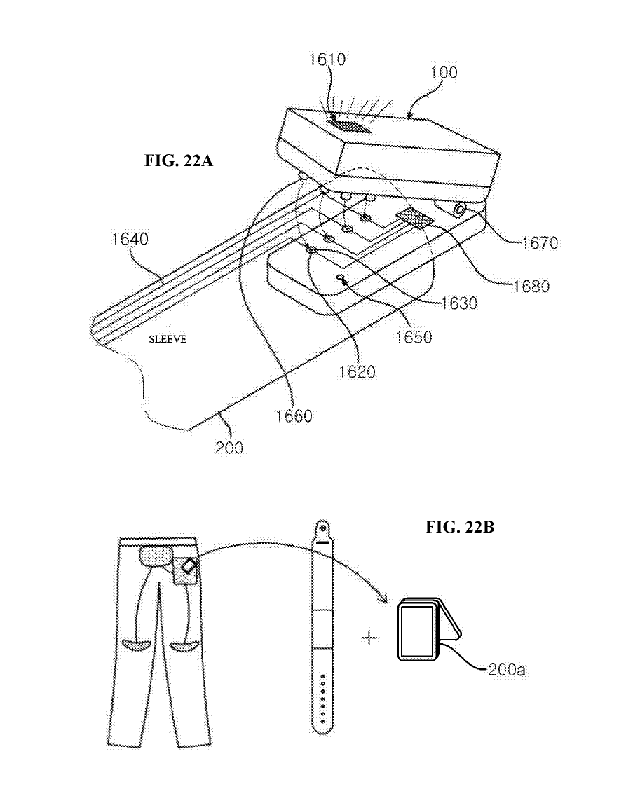

FIGS. 22A and 22B are diagrams illustrating a configuration, in which the mobile device 200, which is configured to be detachable in a clip form, is coupled with the fabric device 100, according to the embodiments as disclosed herein.

Referring to the FIG. 22A, the mobile device 200 is, for example, a main body of a smart watch which does not have a strap or is separable from the strap. The mobile device 200 in the clip form may also be a smart watch in a clip form, and may also be one, in which a main body of a smart watch is combined with a case in a clip form. The mobile device 200 in the clip form includes connection terminals 1620 and 1660 so as to be connected with the fabric device 100.

When the mobile device 200 is in contact with or in proximity to the sleeve of the fabric device 100, a contact or proximate sensor 1650 may display whether the mobile device 200 is in contact with or in proximity to the fabric device 100 and is connected with the fabric device 100 through an output means such as a display 1610 or a speaker (not illustrated). The connection terminal 1660 may be, for example, one electrode of a USB connection terminal supported by the mobile device 200. When the clip of the mobile device 200 is closed, the connection terminal 1660 of the mobile device 200 is connected with the connection terminal 1630 of the fabric device 100 and the connection terminal 1620 at a lower side of the clip. The main body of the smart watch in the clip form assigns intensive elasticity to a hinge part 1670, and thus the main body of the smart watch may maintain strong contact with the connection terminal of the garment device in an engagement state.

The mobile device 200 including the main body in the clip form may be mounted at various positions of the fabric device, in addition to the sleeve part. For example, the main body 200a in the clip form may also be incorporated in a pocket of trousers as illustrated in the FIG. 22B.

In this case, a controller 1680 of the mobile device 200 may be configured to perform the control of the fabric device 100.

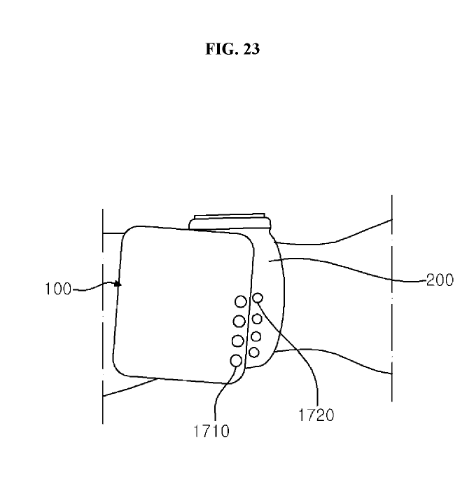

FIG. 23 is a diagram illustrating a connection terminal 1710 of the fabric device 100 and a connection terminal 1720 of a strap of the mobile device 200 which are implemented in forms of a protrusion and a fitting hole respectively, according to the embodiments as disclosed herein.

In an embodiment, a strap of the mobile device 200 includes the connection terminal 1720 in a form of a fitting hole. The fabric device 100 includes the connection terminal 1710 in a form of a protrusion. The connection terminal 1710 in the form of the protrusion is coupled with the connection terminal 1720 in the form of the fitting hole while being inserted into connection terminal 1720 so that the mobile device 200 and the sleeve of the fabric device 100 are fixed while being connected with each other. In this case, the number of connection terminals 1720 of the mobile device 200 may be changed according to a connection standard. For example, in the case of a form conforming to the USB standard, the mobile device 200 may include the four fitting holes 1720.

In an embodiment, a different command signal may be generated according to the number of fittings or a fitted position of the connection terminal 1710 in the form of the protrusion and the connection terminal 1720 in the form of the fitting hole. For example, when the strap includes the four connection terminals 1720 in the form of the fitting hole and the fabric device 100 includes the two connection terminals 1710 in the form of the protrusion, the mobile device 200 may recognize a case where the protrusions 1710 are fitted to the number 1 and 2 fitting holes and a case where the protrusions 1710 are fitted to the number 3 and 4 fitting holes as different connection modes. According to a connection mode, the mobile device may perform various controls such as a display of different screens or performance of different applications.

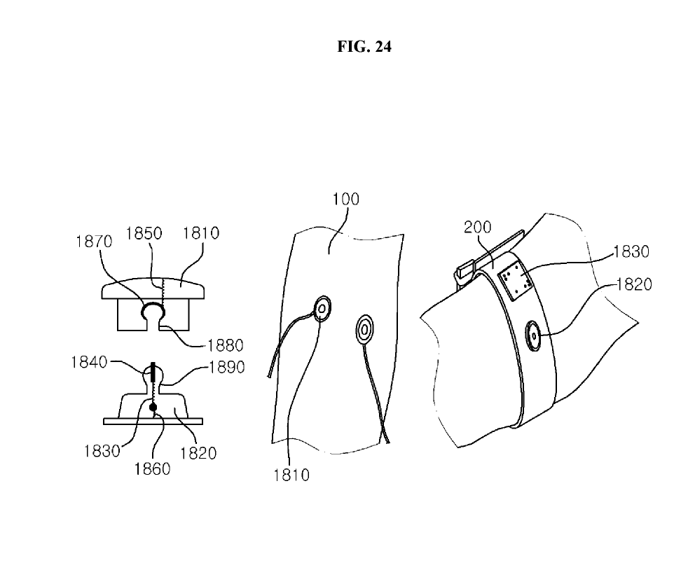

FIG. 24 is a diagram illustrating connectors 1810 and 1820 provided with connection terminals 1860 and 1870, which are in a form of a snap button, and have improved coupling force by an elastic member 1830, according to the embodiments as disclosed herein. In an embodiment, the connector 1810 in a form of a female snap button includes a fitting part 1880 and a connection line 185. The fitting part 1880 includes the connection terminal 1870. The connector 1820 in a form of a male snap button includes a protrusion 1890 and a connection line 1860. The protrusion 1890 includes the connection terminal 1840 and the elastic member 1830. When the two connectors 1810 and 1820 are coupled, the elastic member 1830 enables the two connection terminals 1870 and 1840 to maintain an electrical connection state through physical pressure.

In the present exemplary embodiment, the fabric device 100 includes the connector 1810. A strap part of the mobile device 120 includes the connector 1820. The strap of the mobile device 120 may further include a separate sensor 1830. The sensor 1830 may include, for example, a heat rate sensor (or a part of the sensor) or a proximate sensor.

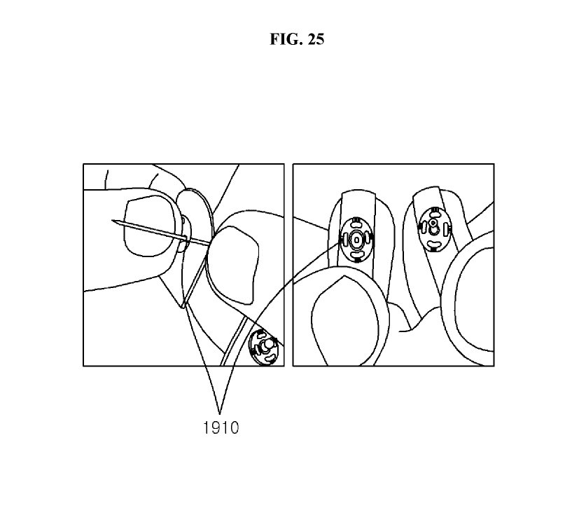

FIG. 25 is a diagram illustrating a connection of the connector 1810 of the FIG. 24 to a garment device and a conductive thread, according to the embodiments as disclosed herein.

A hole of a button is connected with the connection line 1850 of the FIG. 24 and a conductive thread 1910. For example, a thread weaved while a non-conductive thread crosses the conductive thread 1910 may be used. Through the connection, a garment device 110 may be electrically connected with the connector 1810. In the present exemplary embodiment, it is illustrated that the garment device 110 is connected through the connector 1810 in the form of the snap button, but a contact region/state coupled when the button is fastened may be changed according to a position of the button, the kind of button, and the configuration of the conductive thread, and even within the same garment device, it is possible to discriminate a function by an operation of fastening each button.

For example, various command signals, such as a command to determine a target to be connected among the external devices, which are in contact with or in proximity to the garment device. For example, when a sleeve of the fabric device 100 includes the button, the connected mobile device 200 may be recognized as a smart watch. When a pocket includes the button and the button is connected, the connected mobile device 200 may be recognized as a smart phone or a battery. Otherwise, when the mobile device is connected with the button at a lower side of the back of the fabric device 100, the fabric device 100 may be recognized as being connected with a charging device.

Accordingly, the labels are names of various devices or constituent elements are discriminatingly used as “a first . . . ”, a second . . . ”, or the like, and it is apparent that the devices or a constituent element are limited to by the terms. The terms are simply used for discriminating one device or constituent element from another device or constituent element. Accordingly, a first device or constituent element mentioned below may be a second device or constituent element within the technical spirit of the present invention.

The present invention is not limited to the above specific preferred example embodiments, the example embodiments may be variously modified by those skilled in the art to which the present invention pertains without departing from the subject matters of the present invention claimed in the claims, and the modifications belong to the scope disclosed in the claims.

The foregoing description of the specific embodiments will so fully reveal the general nature of the embodiments herein that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the technical spirit and scope of the embodiments as described herein.

1. (canceled)

2. (canceled)

3. (canceled)

4. (canceled)

5. (canceled)

6. (canceled)

7. (canceled)

8. A smart interaction system, comprising:
a fabric device comprising a conductive thread, a sensing area comprising a partial area of the conductive thread, and a sensor; and
a control module of a mobile device configured to be detachably connected to the fabric device and to communicate with the fabric device,
wherein the sensing area comprises a plurality of the conductive threads of the fabric device, wherein a touch input provided to the sensing area is communicated to a touch interface on the mobile device, and
the touch interface on the mobile device is driven by the touch input to the sensing area.

9. The smart interaction system of claim 16, wherein the touch interface includes a first area and a second area, and the sensing area includes a third area physically corresponding to the first area and a fourth area physically corresponding to the second area; and
wherein the touch input sensed in the third area is recognized as an input to the first area of the touch interface, and the touch input sensed in the fourth area is recognized as an input to the second area of the touch interface.

10. The smart interaction system of claim 16, wherein the touch interface includes a first area coupled to a function in a first mode of the mobile device and a second area which is different from the first area and coupled to the function in a second mode;
wherein the sensing area includes a third area physically corresponding to the second area;
wherein when the mobile device connected to the fabric device, the mobile device is operated in the first mode and when the mobile device disconnected to the fabric device, the mobile device is operated in the second mode; and
wherein the touch input sensed in the third area is recognized as an input to the second area of the touch interface in the second mode.

11. (canceled)

12. (canceled)

13. (canceled)

14. (canceled)

15. (canceled)

16. The smart interaction system of claim 8, wherein the sensing area of the fabric device communicates with the mobile device by using the conductive thread.

17. The smart interaction system of claim 8, wherein the sensing area has conductive threads and non-conductive threads wherein the non-conductive threads are intervened between the conductive threads and prevent the conductive threads from being electrically connected.

18. The smart interaction system of claim 17, wherein a signal based on the touch input for one axis is transmitted to the mobile device.

19. The smart interaction system of claim 17, wherein a signal based on the touch input for two axes is transmitted to the mobile device.

20. The smart interaction system of claim 8, wherein the sensing area of the fabric device comprises a plurality of conductive threads in a stitched pattern.
